(19) United States
(12) Patent Application Publication
(10) Pub. No.: US 2010/0292333 A1
(43) Pub. Date: Nov. 18, 2010

(54) COMPOSITIONS SUITABLE FOR THE TOPICAL TREATMENT OF FUNGAL INFECTIONS OF THE SKIN AND NAILS

(75) Inventor: Peter Mladenovich, Oakville (CA)

Correspondence Address: Heenan Blaikie LLP;Bay Adelaide Centre, 333 Bay Street, Suite 2900, P.0. Box 2900, Toronto, ON, M5H 2T4, CA

(73) Assignee: bioCEPTA Corporation

(21) Appl. No.: 12/466,615

(22) Filed: May 15, 2009

(51) Int. Cl. A61K 31/192; A01N 37/10; A61P 31/10

(52) U.S. Cl. 514/568

(57) ABSTRACT

The present invention relates to pharmaceutical compositions suitable for the topical treatment of fungal infections of the skin and nails, such as tinea pedia and tinea cruris, among others. The active ingredients of the present compositions are low molecular weight organic acids and their salts, many of which are previously known as antifungal agents to some degree but which, when used in combination, produce a synergistic enhancement in antifungal potency such that the combined effect is greater than that when used by the agents alone. The antifungal activity of the active ingredients is further enhanced through the use of non-volatile hygroscopic solvents rather than the aqueous or volatile organic solvents used in the art.

### FIELD OF THE INVENTION

The present invention relates to compositions suitable for the topical treatment of fungal infections that may develop on the skin (dermatomycoses) as well as toe and finger nails (onychomycosis). These fungal infections, also commonly known as Tinea pedis (athlete's foot), Tinea unguium (nail infections), Tinea cruris (Cock itch), Tinea corporis, Tinea versicolor and Tinea candidiasis, among others, are caused by different types of fungus such as those of the gena Trichophyton, Epidermophyfon, Microsporum and Candida.

### BACKGROUND OF THE INVENTION

Fungal infection of the nails is one of the most common diseases of the nail bed or plate. It is estimated that between 6 and 8 percent of the adult population is affected by such fungal infections to a varying degree. Fungal infections of the nail (onychomycosis) are often caused by dermatophytic fungi, most often by one of Trichophyton mentagrophytes (also known as Tichophyton interdigitale) and Trichophyton rubrum, although numerous other fungi are also known to cause such infections. Fungal infection of the skin (dermatomycoses) such as Tinea pedis (athlete's foot), Tinea cruris Cock itch) and Tinea corporis are also commonly caused by T. rubrum and T. mentagrophytes, among other fungi. T. mentagrophytes causes a web or vesicular infection and is generally easily treated by conventional over-the-counter (OTC) medications. T. rubrum, however, causes a more wide-spread infection and is significantly more difficult to treat.

A large number of pharmaceutical compositions for use in the topical treatment of skin and nail fungal infections have been described in the art. These topical applications include lotions, sprays, gels and ointments containing a variety of prescription and non-prescription active ingredients.

U.S. Pat. No. 6,080,744 describes cream-based topical treatments for mycotic infections consisting of a blend containing a multitude of active ingredients including ketaconazole, nystatin, miconazole nitrate, tolnaftate, clotrimazole, undecenoic acid, zinc undecenoate, propionic acid and sodium propionate. Optionally, the compositions may include additional active ingredients such as an antibacterial agent (e.g., gentamicin) or an anti-inflammatory agent (e.g., dipropionate betamethasone).

European Patent Application 1 787 652 A1 describes an antifungal composition comprising Melaleuca alternifolia essential oil (consisting of terpinene-4-ol and cineole), a benzoate compound (benzoic acid and its salts) and the known antifungal compound miconazole nitrate.

U.S. Pat. No. 5,512,200 describes compositions having a low pH comprised of a two inorganic acids (one dissociating completely in water, the second less strong than the first) and two organic acids. Among the uses taught for these compositions are pharmaceutical agents, water treatment, topical disinfectants, and the replacement of battery fluids. Owing to the low pH of these compositions, they would be expected to be particularly irritating to the skin upon topical administration, leading to problems with patient compliance and thus effective eradication of the infection.

U.S. Pat. No. 6,664,292 describes compositions for the treatment of fungal infections of the nail comprised of a lower alcohol, preferably methanol, and a lower carboxylic acid.

U.S. Pat. No. 4,824,865 describes compositions containing 2-hydroxyoctanoic acid, 2-ketooctanoic acid and certain esters thereof for the treatment of various skin conditions, including tinea pedis, in vehicles such as aqueous or non-aqueous ethanol.

U.S. Pat. No. 6,214,889 describes liquid and gel compositions for the treatment of adverse skin conditions consisting of one or more of potassium, sodium, calcium or cesium formate, preferably in concentrations of about 50% in distilled water and an optional gelling agent.

U.S. Pat. No. 6,921,529 describes a topical composition containing a weak organic acid dispersed in a polymeric matrix to form a supersturated hydro-gel. Optionally, the use of other, known, antimycotic agents such as azole derivatives (such as ketaconazole, miconzaole nitrate, clotrimazole), undecylenic acid, tea tree oil (Melaleuca alternifolia), or salicyclic acid may be added to the hydrogel.

International Patent Application 2006/042324 describes non-water soluble, film-forming compositions which adheres to body tissues comprising alkyl and hydroxyalkyl celluloses, a polar protic solvent, an antifungal agent such as naftidine, ciclopirox or terbinafine, a glycol ether, an antipruritic agent, and one or more of a solubility enhancing, surfactant and wetting agent.

U.S. Pat. No. 6,159,977 describes antifungal compositions comprising an antifungal agent such as clotrimazole, toinaftate, nystatin or undecylenic acid in combination with dimethylsulfoxide and an antiinflammatory agent, in an anhydrous solution of polyglycol.

U.S. Pat. No. 7,074,392 describes antifungal compositions comprising an antifungal agent selected from common agents such as naftidine, miconazole, clotrimazole, etc., and optionally a keratolytic agent, a humectant, water, a polymeric film-forming agent such as a hydrophobic water-insoluble polymer, and a solvent system involving at least one volatile solvent. Optionally, the compositions may also include one or more of an antibacterial, antiviral or antipsoriatic agent.

U.S. Pat. No. 6,231,875 describes compositions comprising an active ingredient, an acidifier (acids with low pH such as hydrochloric acid, sulfuric acid, glycolic acid, lactic acid or acetic acid), and a volatile solvent (e.g., alcohols, acetone, ethyl acetate). Active ingredients are described as preferably being antifungal drugs such as azoles (ketoconazole, fluconazole, clotrimazole), allylamines (terbinafine or naftifine), or mixtures therefore, and may also include antibacterial agents, antipsoriatic drugs, nail growth promoters, and nutrients.

Although they are present in topical antifungal compositions described in the art, a number of agents known to exhibit an antifungal property to some degree have been found to be not safe and effective by the US Food & Drug Administration (FDA) when used as active ingredients in OTC topical antifungal preparations. These agents include alcdoxa, alum, aluminum sulfate, secondary amyltricresols, basic fuchsin, benzethonium chloride, benzoic acid, benzoxiquine, boric acid, camphor, candicidin, chlorothymol, coal tar, dichlorophen, menthol, methylparaben, oxyquinoline, oxyquinoline sulfate, phenol, phenolate sodium, phenyl salicylate, propionic acid, propylparaben, resorcinol, salicylic acid, sodium borate, sodium caprylate, sodium propionate, sulfur, tannic acid, thymol, tolindate, triacetin, zinc caprylate, and zinc propionate. Currently, the only antifungal agents deemed safe and effective by the FDA for use as active ingredients in OTC topical antifungal preparations are clioquinol, haloprogin, miconazole nitrate, povidone-iodine, tolnaftate, undecylenic acid and its salts, and clotrimazole. In addition to over-the-counter medications, numerous prescription medications, such as terbinafine, are also available to treat fungal infections. While terbinafine is available in various topically applied OTC formulations, they are not indicated for use in the treatment of nail infections as is its orally administered form.

Although pharmaceutical compositions using the some of the above ingredients now viewed by the FDA as not safe and effective are known and have previously been sold as over-the-counter medicaments before the FDA decision regarding their efficacy, most notably Whitfield's Ointment (a combination of benzoic and salicylic acids in an emulsifying base), they are generally regarded as ineffective. While seen as providing some relief of symptoms, relapse of the infection is almost universal following cessation of treatment with such compositions.

While a large number of different topical preparations, both prescription and non-prescription, are available for the treatment of fungal infections, most of these preparations do not have a wide enough spectrum effectively to treat the different types of persistent fungi that infect the skin and nails, in particular, fungal infections of the nail, which are often embedded deep within the nail and therefore difficult to reach with topical treatments. Additionally, some of the existing preparations contain toxic and allergenic substances, making treatment intolerable to the subject. These combined factors can lead to a treatment period that may be significantly protracted and which may not be able completely to eradicate the infection owing either to ineffectiveness or noncompliance with the administration guidelines.

As a result, there remains a need for new, inexpensive compositions that exhibit an antifungal effect sufficient to eradicate infections rather than providing a temporary respite in which infections reoccur following the cessation of treatment.

### SUMMARY OF THE INVENTION

The present compositions are suitable as broad spectrum, topical antifungal preparations for the treatment of a variety of fungal infections that may develop on the skin and nails, or which may be present and viable on surfaces which may come in contact with skin and nails. As a result, the compositions of the present invention may be used either therapeutically to treat a pre-existing infection, or as a fungicidal disinfectant to cleanse surfaces that may harbor the fungus, thereby preventing or limiting the occurrence of infections. Compositions of the present invention are comprised of a combination of low molecular weight organic acids, or their derivatives which revert to organic acids in the presence of moisture, ideally with the acids being in salt form, dissolved in a non-volatile hygroscopic carrier.

Although many of the carboxylic acids used in the present compositions have been previously known to possess fungicidal properties to varying degrees, they have been found generally not to be effective for use as the active ingredients in over-the-counter antifungal products. However, it has now been found that when used in combination, rather than as individual agents, the combination provides a synergistic enhancement of the individual antifungal effects of each component. This synergistic effect has allowed for the preparation of antifungal preparations having a broad spectrum antifungal effect without requiring the presence of common synthetic fungicidal agents, such as miconazole, clotrimazole and terbinafine, thereby reducing the cost of the preparations as well as avoiding the need for the use of prescription medicines.

As described in further detail below, a preferred composition of the present invention comprises the combination of sodium formate, zinc propionate, calcium propionate, and sodium benzoate in the hygroscopic carrier propylene glycol, with either glycerol (for a liquid formulation) or hydroxy ethyl cellulose (for a gel formulation).

The present invention is the result of the inventor's personal experience with a fungal foot and toe infection contracted at a fitness club, a common source of such infections. The fungal foot and toe infection was diagnosed as tinea pedis (athlete's foot) and treatment with several commercial antifungal preparations in powder and liquid forms was attempted. While the commercial preparations, including Dr. Scholl's®, used to treat the fungal infection did alleviate the condition significantly, extended treatments were not able successfully to eradicate the infection. Moreover, available treatments often led to cracking of the skin, resulting in deeper infections following the return of symptoms. As a result of the failure of the available commercial preparations, some of which were irritating and inflammatory to the surrounding tissue, to eradicate the infection, alternative treatments possessing a broader anti-fungal spectrum were sought, thus leading to the present invention.

The active ingredients useful in the present compositions are combinations of two or more low molecular weight organic acids and their salts. Typical low molecular weight organic acids include both substituted and non-substituted aliphatic (saturated and unsaturated) and aromatic acids. Ideally, the organic acid has less than 15, and preferably less than 10 carbons, with the longest carbon chain being less than 8 carbons in length. The organic acids used in the present compositions can possess as substituents one or more functional groups, such as alkyl, alkenyl, alkynyl, halogen, hydroxy, carbonyl, carboxylic acid, aldehyde, ester, amide, carbonate, carbamate, ether, amino, cyano, isocyano, oxy, oxo, thia, aza, azide, imine, nitro, nitrate, nitroso, nitrosooxy, cyanate, isocyanate, thiocyanate, isothiocyanate, sulfinyl, sulfhydryl, sulfonyl, phosphino, wherein each of the alkyl, alkenyl, alkynyl and amino groups may themselves be optionally substituted with one or more of the preceding functional groups. Of particular interest are those functional groups, such as hydroxy, which will impart or augment a hygroscopic character to the acid. Also useful in the compositions are organic acids containing multiple carboxylic acid groups.

Preferably, the organic acids will be selected from branched and unbranched alkanoic acids, hydroxyalkanoic acids, alkenoic acid, aromatic acids, and hydroxyaromatic acids. Such organic acids include formic, acetic, propionic, butyric, valeric, caproic, enanthic, caprylic, glyceric, glycolic, lactic, tartaric, gluconic, benzoic, mandelic, salicylic, acrylic, acetoacetic, pyruvic, adipic, aldaric, fumaric, glutaric, maleic, sorbic, malic, malonic, oxalic, succinic, tartronic, citric, isocitric, aconitic, and carballylic acids, as well as their further branched and substituted derivatives. In addition to carboxylic acids, the corresponding sulfonic and phosphonic acid derivatives may also be used in the present compositions. Particularly preferred acids for use in the present compositions are formic, propionic and benzoic.

In addition to the use of the free acids, easily hydrolyzable anhydride, mixed anhydride and ester derivatives of the acids may also be used, it being understood that such derivatives would revert to their acid form during treatment through their exposure to moisture.

To add to the hygroscopicity of the organic acids as well as to raise the pH of the otherwise acidic compositions, it is preferable to use the acids in their salt forms.

The salts of organic acids useful in the present compositions are preferably metal salts. More preferably, the metal salts are those of alkali metals (e.g., lithium, sodium and potassium), alkaline earth metals (e.g., magnesium and calcium) and metals exhibiting amphoteric properties (e.g., aluminum and zinc). Particularly preferred salt forms to be used in the present compositions are sodium, potassium, calcium and zinc. Alternatively, if it is desired that the compositions be prepared with the free acids, a buffering agent, such as sodium, potassium or calcium bicarbonate or other such agent, may be used to form salts of the carboxylic acids in situ.

Preferred active ingredients for the compositions of the present invention are sodium formate, zinc propionate, calcium propionate and sodium benzoate.

The active ingredients of the present compositions are dissolved in a carrier comprised primarily of one or more non-volatile hygroscopic solvents. The composition is, of course, nontoxic to humans and compatible with the skin. Such solvents preferably include glycerine, diglycerol, ethylene glycol, propylene glycol, sorbitol, xylitol, maltitol, low molecular weight polyglycols, such as polyethylene glycol or polypropylene glycol with molecular weight less than 500 gmol⁻¹, among others, and their derivatives, particularly monoether and ester derivatives. Most preferred as solvents are glycerine and propylene glycol. The use of the aforementioned hygroscopic solvents rather than water and lower monoalcohols such as methanol and ethanol, which are commonly used in topical antifungal preparations, has been found to be beneficial owing to their lower rates of evaporation, thereby allowing for extended contact times with the infected area. When evaporation of the solvent occurs too readily, the active ingredients of the compositions can precipitate, forming a powder, which is easily dispersed from the treatment site, making treatment less effective.

Also usable as a solvent, preferably as a co-solvent in a lesser amount, are ester derivatives of the previously mentioned hydroxy-containing solvents, such as di- and tri-glycerides, wherein the acid component is one of the previously mentioned carboxylic acids.

The carrier system used for the present compositions will preferably have a low to negligible water content. Such compositions demonstrate increased potency over compositions in which the carriers have higher water content.

The combination of acids in the present compositions may be added to the carrier in any amount desired, ideally from about 0.1% to about 50% total acid content by weight of the composition, the lower limit being to provide a sufficient effect for a given combination for the intended application and the upper limit being the saturation point of a given carrier system. Preferably, the total acid content is about 5% to about 30% by weight of the composition, and more preferably the total acid content is from about 10 to about 20% by weight of the composition. Within the total acid content, the individual acid components may be distributed in any proportion desired although it is preferable that any one acid does not comprise more than about 75% of the combination. Preferably, each acid is present to the extent of about 2% to about 10% by weight of the total formulation.

If desired, the viscosity of a formulation can be altered depending upon the properties exhibited by a particular mixture of acids/salts and solvent, and the intended method of treatment. This may be accomplished through the addition of one or more thickening agents such as soluble cellulose derivatives, oligosaccharides, polysaccharides, polyethylene glycol or polypropylene glycol with molecular weight greater than 1000, or through the use of colloidal suspensions such as silicon dioxide and hydrated aluminum oxide. Such agents are particularly useful for the preparation of gels and pastes, thereby allowing for longer retention at the treatment site. Preferred thickening agents are hydroxy ethyl cellulose and Aerosil® 200 (a fumed silica).

The compositions of the present invention exhibit antifungal properties and are therefore useful in the topical treatment against fungi which cause various infections of the skin and nails such as tinea unguium, tinea pedis, tinea cruris, tinea corporis, tinea versicolor and tinea candidiasis. The fungi causing these tinea infections are known commonly to include the various species of Trichophyton, Epidermophyton and Microsporum, as well as Candida. The compositions are particularly useful in treating nail infections, which are often caused by T. mentagrophytes (also known as T. interdigitale) and T. rubrum. Fungus infection of the skin such as tinea pedis, tinea cruris and Tinea corporis are also known to be commonly caused by T. rubrum and T. mentagrophytes and therefore are also preferably treated with the present compositions.

As well as being applied directly to the infected area, compositions of the invention can also be pre-applied to bandages or other absorbent material, which is then applied to the infected area. Alternatively, the treated area can be wrapped in a bandage or other protective covering.

In addition to the treatment of fungal infections of the skin and nail, the compositions of the present invention may be used as a fungicidal disinfectant to cleanse surfaces that may harbour the infecting fungus, thereby preventing or limiting the occurrence of infections. Compositions of the invention, preferably ones with low viscosity, can be used as a fungicidal disinfectant for skin and surfaces which frequently come into contact with the skin such as door knobs, shopping cart handles, public showers and changing rooms.

Thus, in accordance with the invention, methods of treatment of fungal infections of the skin and nails have been provided wherein a therapeutically effective amount of the compositions described herein is applied to the infected area. As well, the use of the compositions described herein for the topical treatment of fungal infections of the skin and nails has also been provided. Further, the use of the compositions described herein in the preparation of a medicament for the topical treatment of fungal infections of the skin and nails is also provided.

### EXEMPLARY FORMULATIONS OF THE ANTIFUNGAL COMPOSITIONS OF THE INVENTION

Formulation of the antifungal compositions of the present invention may be prepared by any means known to the skilled person. Two exemplary, but non-limiting, formulations of the present invention, a liquid and a gel formulation, are provided below. In addition to these exemplary formulations, other topical formulations, prepared using methods known to the skilled person, may also be used for the administration of the antifungal compositions of the present invention.

### Example 1

### Liquid Formulation

A preferred liquid formulation contains the following ingredients;

| Ingredient | % by Wt. |
|---|---|
| Propylene glycol | 72 |
| Sodium benzoate | 5 |
| Calcium propionate | 5 |
| Zinc propionate | 5 |
| Sodium formate | 3 |
| Glycerol | 10 |

This formulation may be prepared by, among other methods, first warming the propylene glycol and sequentially dissolving in it, the sodium benzoate, calcium propionate, zinc propionate and sodium formate. After dissolution of each of the salts, the glycerol is added, following which the mixture is cooled to room temperature.

### Example 2

### Gel Formulation

A preferred gel formulation contains the following ingredients:

| Ingredient | % by Wt. |
|---|---|
| Propylene glycol | 80 |
| Sodium benzoate | 5 |
| Calcium propionate | 5 |
| Zinc propionate | 5 |
| Sodium formate | 3 |
| Hydroxy ethyl cellulose | 2 |

This formulation may be prepared by, among other methods, first warming the propylene glycol and sequentially dissolving in it the sodium benzoate, calcium propionate, zinc propionate and sodium formate. After dissolution of each of the salts, the hydroxy ethyl cellulose is slowly added to prevent agglomeration, following which the mixture is cooled to room temperature.

In addition to the exemplary preparations described herein, the formulations may also be prepared by first dissolving the carboxylic acids and/or their salts and derivatives in water, or other relatively low boiling aqueous or non-aqueous solvents and, following the addition of this initial mixture to the carrier system to remove all or the majority of the water or low boiling solvent through heating or desiccation of the composition. This procedure can assist in the initial dissolution of the carboxylic acid components.

### Use and Efficacy of the Antifungal Compositions of the Invention

In general, the antifungal compositions are suitable to be used to treat fungal infections on the skin and nail by direct application of the composition on the infected area using, for example, a liquid or gel formulation, such as the formulations in Examples 1 and 2. Liquid preparations are particularly beneficial for infections that spread under skin folds or in narrow crevasses, such as nail beds. Gel preparations are particularly useful for open skin and nail infections which can additionally be covered by a protective film such as a cloth, glove, bandage, or wrap-around tape.

### Application of the Antifungal Compositions to Infected Nails

The antifungal efficacy of the invention was initially demonstrated by the inventor during development of the preparation for personal treatment of an “athlete's foot” infection, which subsequently spread to the nails. The inventor and many other individuals have tested compositions of the invention, experiencing eradication of the infection without reoccurrence following the completion of treatment.

In two individuals, each with fungal infections of the nail on all nails of both feet, the nails were first debrided to remove damaged nail material. Following debridement, the liquid formulation (as described in Example 1) was applied to each nail and the surrounding skin twice daily (morning and evening). Within two weeks, both individuals perceived a marked reduction in the sensation of irritation at the nails. Treatment was continued for eight weeks at which time cultures taken from one individual tested negative for nail fungus. Following cessation of treatment, reoccurrence of the infection was not reported by either individual. No irritation or other discomfort owing to the application of the invention was reported by either subject.

In two individuals, each with a fungal infection of the nail on a big toe, the liquid formulation (as described in Example 1) was applied twice daily (morning and evening) for eight weeks at which there was no evidence of infection. Prior to treatment the nails were not debrided. Following cessation of treatment, reoccurrence of the infection was not reported by either individual. No irritation or other discomfort owing to the application of the invention was reported by either subject. One of the two individuals was also suffering from a fungal infection of the skin on the top and underside of the foot and between the toes. The gel formulation (as described in Example 1) was applied to this area twice daily. Although treatment was continued for eight weeks, in conjunction with treatment of the infected nail, the redness and irritation had cleared within two days.

One individual with a fungal infection on both feet between the toes was treated twice daily (morning and evening) with the liquid formulation (as described in Example 1) for one week. At the end of treatment the infection had been eradicated and no reoccurrence was reported. No irritation or other discomfort owing to the application of the invention during treatment was reported.

One individual with fungal infections on the under arms and the back sides of the elbows was treated with the liquid formulation (as described in Example 1) twice daily (morning and evening) for five days. Following cessation of treatment, both infections had been completely eradicated; no reoccurrence was reported. No irritation or other discomfort owing to the application of the invention during treatment was reported.

While the treatment of infected nails in the above examples relied upon an eight-week application period, this should not be taken as the minimum time required for successful eradication of nail infections. An eight-week period was selected based upon the treatment periods generally indicated for fungal nail infections. Minimum effective treatment times using the present compositions may be determined in a formalized clinical trial program designed for such an outcome.

The combination of organic acids used in the present compositions are believed to exhibit a synergistic effect based on initial attempts to treat fungal infections using an aqueous solution of calcium and sodium propionate (constituents of the known antifungal agent Mycoban®, which is commonly used as a food preservative). Although this treatment led to an initial clearing of fungal infections of the skin, the infections reoccurred within two weeks following cessation of treatment. In contrast, the infection could be eradicated when using a composition of the present invention combining as few as two organic acids, in particular salts of propionic acid and benzoic acid, in propylene glycol. This increased effect was further enhanced through the addition of a third organic acid.

### Confirmation of the Antifungal Activity of the Antifungal Compositions—Liquid Broth Assay

The antifungal activity of the present compositions was confirmed through in vitro testing using an antifungal microdilution method used in measuring the antifungal susceptibility of filamentous fungi that cause invasive infections. Fungal colonies are grown on potato glucose agar (PGA). One colony is picked and grown in Sabouraud glucose broth (SGB) at 24° C. for 3 days in the presence of test compound. Microdilution trays are incubated at 24° C., and are read after 5 days of culture. Turbidity in the microdilution wells is scored with the aid of a reading mirror and compared with that of the growth control. A numerical score from 0 to 4 is given to each well using the following scale: 0=optically clear or absence of growth, 1=slight growth (25% of growth control), 2=prominent reduction in growth (50% of growth control), 3=slight reduction in growth (75% of growth control), 4=no reduction in growth.

The turbidity scores (average of 3 tests) for T. rubrum and T. mentragrophytes grown in different concentrations (or dilutions) of test compounds for 5 days was as follows:

Trichophyton rubrum

Purity (%)

| | 0 | 0.625 | 1.25 | 2.5 | 5 | 10 |
|---|---|---|---|---|---|---|
| Formulation | 4 | 0 | 0 | 0 | 0 | 0 |
| of Ex. 1 | | | | | | |
| Carrier | 4 | 4 | 4 | 4 | 2.67 | 1 |

Trichophyton mentagrophytes

Purity (%)

| | 0 | 0.625 | 1.25 | 2.5 | 5 | 10 |
|---|---|---|---|---|---|---|
| Formulation | 4 | 1 | 1 | 0.67 | 0 | 0 |
| of Ex. 1 | | | | | | |
| Carrier | 4 | 4 | 4 | 4 | 3 | 2 |

This testing demonstrates that while the tested formulation was active against both T. mentagrophytes and T. rubrum, it exhibited a more potent antifungal against T. rubrum, which is generally more difficult to treat than T. mentagrophytes.

As many changes can be made to the provided examples without departing from the scope of the invention, it is intended that all material herein be interpreted as illustrative of the invention and not in a limiting sense.

1. A non-toxic composition compatible with the skin comprising a combination of two or more low molecular weight organic acids or their salts, or organic acid derivatives which revert to the organic acids in the presence of moisture, dissolved in a carrier, wherein
the low molecular weight organic acids may be selected from:
saturated and unsaturated, cyclic and acyclic, branched and unbranched aliphatic carboxylic acids containing less than 15 carbon atoms and wherein the longest carbon chain has eight carbons,
aromatic carboxylic acids containing fewer than 15 carbon atoms, and
the corresponding sulfonic and phosphonic acid derivatives of said aliphatic and aromatic carboxylic acids,
each of which may be optionally substituted with one or more of alkyl, alkenyl, alkynyl, halogen, hydroxy, carbonyl, carboxylic acid, aldehyde, ester, amide, carbonate, carbamate, ether, amino, cyano, isocyano, oxy, oxo, thia, aza, azide, imine, nitro, nitrate, nitroso, nitrosooxy, cyanate, isocyanate, thiocyanate, isothiocyanate, sulfinyl, sulfhydryl, sulfonyl, and phosphino functional groups, wherein each of the alkyl, alkenyl, alkynyl and amino groups may themselves be optionally substituted with one or more of the preceding functional groups;
the carrier is comprised of one or more non-volatile hygroscopic solvents; and
wherein the combination of low molecular weight organic acids comprises 0.1% to 50% by weight of the composition, and no single acid or salt comprises more than 75% by weight of the total acid content.

2. The composition of claim 1 in which the organic acids are selected from the group consisting of formic, acetic, propionic, butyric, valeric, caproic, enanthic, caprylic, glyceric, glycolic, lactic, tartaric, gluconic, benzoic, mandelic, salicylic, acrylic, acetoacetic, pyruvic, adipic, aldaric, fumaric, glutaric, maleic, sorbic, malic, malonic, oxalic, succinic, tartronic, citric, isocitric, aconitic, and carballylic acids, as well as their substituted derivatives and derivatives which revert to the organic acid in the presence of moisture.

3. The composition of claim 2 in which one or more of the organic acids are used in a salt form.

4. The composition of claim 3 in which the organic acids are used in a salt form selected from alkali metals, alkaline earths, aluminum, or zinc.

5. The composition of claim 3 in which the salts of the low molecular weight organic acids are formate salts, propionate salts, and benzoate salts.

6. The composition of claim 5 in which the salts of the low molecular weight organic acids are sodium formate, calcium propionate, zinc propionate and sodium benzoate.

7. The composition of claim 1 in which each of the organic acids or their salts are present in amounts of 3 to 7% by weight.

8. The composition of claim 1 in which the carrier comprises a hygroscopic solvent selected from polyhydroxylated solvents and polyglycols having molecular weights of less than 500 gmol⁻¹.

9. The composition of claim 8 in which the carrier comprises a hygroscopic solvent selected from the group consisting of glycerine, diglycerol, ethylene glycol, propylene glycol, sorbitol, xylitol, maltitol, and low molecular weight polyglycols, such as polyethylene glycol or polypropylene glycol with molecular weights of less than 500 gmol⁻¹.

10. The composition of claim 9 in which the carrier comprises propylene glycol.

11. The composition of claim 1 which additionally comprises a viscosity increasing agent.

12. The composition of claim 11 which comprises a viscosity increasing agent selected from the group consisting of soluble cellulose derivatives, oligosaccharides, polysaccharides, polyethylene glycol or polypropylene glycol with a molecular weight greater than 1000 g/mol⁻¹, fuming silica and aluminum oxide.

13. The composition of claim 12 which comprises a viscosity increasing agent selected from hydroxy ethyl cellulose and fuming silica.

14. The composition of claim 13 which comprises hydroxy ethyl cellulose as a viscosity increasing agent.

15. A method of treating a fungal infection of the skin or nails comprising administering to a subject a therapeutically effective amount of a topical antifungal composition on the infected area, said composition being non-toxic to the subject and compatible with the skin, and comprising a combination of two or more low molecular weight organic acids or their salts, or organic acid derivatives which revert to the organic acids in the presence of moisture, dissolved in a carrier, wherein
the low molecular weight organic acids may be selected from:
saturated and unsaturated, cyclic and acyclic, branched and unbranched aliphatic carboxylic acids containing less than 15 carbon atoms and wherein the longest carbon chain has eight carbons,
aromatic carboxylic acids containing fewer than 15 carbon atoms, and
the corresponding sulfonic and phosphonic acid derivatives of said aliphatic and aromatic carboxylic acids,
each of which may be optionally substituted with one or more of alkyl, alkenyl, alkynyl, halogen, hydroxy, carbonyl, carboxylic acid, aldehyde, ester, amide, carbonate, carbamate, ether, amino, cyano, isocyano, oxy, oxo, thia, aza, azide, imine, nitro, nitrate, nitroso, nitrosooxy, cyanate, isocyanate, thiocyanate, isothiocyanate, sulfinyl, sulfhydryl, sulfonyl, and phosphino functional groups, wherein each of the alkyl, alkenyl, alkynyl and amino groups may themselves be optionally substituted with one or more of the preceding functional groups;
the carrier is comprised of one or more non-volatile hygroscopic solvents; and
wherein the combination of low molecular weight organic acids comprises 0.1% to 50% by weight of the composition, and no single acid or salt comprises more than 75% by weight of the total acid content.

16. The method of claim 15 in which the composition is comprised of the organic acid salts sodium formate, calcium propionate, zinc propionate, and sodium benzoate.

17. The method of claim 15 in which the fungal infection to be treated is tinea unguium, tinea pedis, tinea cruris, tinea corporis, tinea versicolor, or tinea candidiasis.

18. The method of claim 15 in which the fungal infection to be treated is caused by Trichophyton rubrum or Trichophyton interdigitale.

19. A method of disinfecting a surface susceptible to contamination with a fungus comprising applying to said surface a fungicidally affective amount of an antifungal composition, said composition being non-toxic and compatible with the skin, and comprising a combination of two or more low molecular weight organic acids or their salts, or organic acid derivatives which revert to the organic acids in the presence of moisture, dissolved in a carrier, wherein
the low molecular weight organic acids may be selected from:
saturated and unsaturated, cyclic and acyclic, branched and unbranched aliphatic carboxylic acids containing less than 15 carbon atoms and wherein the longest carbon chain has eight carbons,
aromatic carboxylic acids containing fewer than 15 carbon atoms, and
the corresponding sulfonic and phosphonic acid derivatives of said aliphatic and aromatic carboxylic acids,
each of which may be optionally substituted with one or more of alkyl, alkenyl, alkynyl, halogen, hydroxy, carbonyl, carboxylic acid, aldehyde, ester, amide, carbonate, carbamate, ether, amino, cyano, isocyano, oxy, oxo, thia, aza, azide, imine, nitro, nitrate, nitroso, nitrosooxy, cyanate, isocyanate, thiocyanate, isothiocyanate, sulfinyl, sulfhydryl, sulfonyl, and phosphino functional groups, wherein each of the alkyl, alkenyl, alkynyl and amino groups may themselves be optionally substituted with one or more of the preceding functional groups;
the carrier is comprised of one or more non-volatile hygroscopic solvents; and
wherein the combination of low molecular weight organic acids comprises 0.1% to 50% by weight of the composition, and no single acid or salt comprises more than 75% by weight of the total acid content.

20. The composition of claim 1 which is impregnated within or applied to a bandage or other material to be applied to skin or nails infected by a fungus.
